(19) United States
(12) Patent Application Publication
(10) Pub. No.: US 2008/0035142 A1
(43) Pub. Date: Feb. 14, 2008

(54) Spacer

(76) Inventors: Amar Lulla, Maharashtra (IN); Xerxes Rao, Maharashtra (IN)

Correspondence Address: CONLEY ROSE, P.C., 5601 GRANITE PARKWAY, SUITE 750, PLANO, TX, 75024, US

(21) Appl. No.: 11/577,224

(22) PCT Filed: Oct. 17, 2005
(86) PCT No.: PCT/GB05/03984

§ 371 Date: Jul. 19, 2007

(30) Foreign Application Priority Data
Oct. 15, 2004 (IN) 1113/MUM/2004

(51) Int. Cl. A61M 16/20

(52) U.S. Cl. 128/203.12

(57) ABSTRACT

A spacer device for the oral administration of a volatile medium containing a medicament comprises a chamber (110) having an inlet (102) to admit a measured dose of medicament and an outlet (104) to be received in the mouth, wherein the spacer comprises a butterfly valve (106). Preferably, the chamber comprises two frustoconical members assembled together coaxially at their divergent ends, said inlet and outlet being respectively at the opposed convergent ends.

The present invention relates to a spacer device for the oral administration of a volatile medium containing a medicament.

Spacer devices are attachments to the mouthpiece of an inhaler, particularly for pressurized meter dose inhalers. Various spacer devices are known in the art from a tube spacer with a volume of <50 ml to holding chambers with a volume of up to 750 ml. Generally, spacers are known to reduce coordination difficulties and reduce oropharyngeal deposition thereby considerably increasing the drug delivery in the lungs.

Indian Patent No. 190657 discloses a spacer device for administering orally a volatile liquid composition by inhalation having two conical members made of anti-static material and assembled at their divergent ends. Convergent end of the cone is adapted to receive within it a pumping device of a container filled within the medicinal composition and the convergent end of the other cone adapted to be inserted into the mouth of a patient. The inner surface of the one of said cones is provided with stepped rings and the outer surface of the other cone provided with stepped rings corresponding to the stepped rings in the inner surface. The cones are provided with locking means, such as notch and the projection. Reference can also be made to WO 00/33902.

However, in the spacer device as described in the Indian Patent No. 190657 during accidental exhalation the dose in the spacer chamber gets diluted with the moist breath thereby reducing the efficiency of the drug delivery.

EP 938 908 discloses a cloud chamber (or spacer) having a one-way inspiratory air and one-way expiratory air valving system, which allows repeated breaths to be taken without removal of the device from the mouth of the user. The one-way inspiratory air valve is formed from an elliptically-shaped valve body comprising an elastomeric material having an “X”-shaped cut in the centre portion of the material. During inspiration, the “X”-shaped cut opens, allowing air to be drawn through. U.S. Pat. No. 5,042,467 discloses a spacer having a similar type of valve. However, this document is primarily concerned with providing a medication inhaler incorporating a sonic signalling device comprising an integrally molded plastic body and vibratory reed. A musical tone alerts the user if he is inhaling too rapidly.

An inhalation chamber for use with a metered-dose inhaler is also disclosed in GB 2230456. A face mask adapted to communicate with the nose and/or mouth of an infant or young child communicates with the chamber outlet via a first inhalation valve, and communicates with atmosphere via a second valve permitting exhalation therethrough. The inhalation valve comprises a disc which is biased into a closed position in which it bears against an annular seat by means of a spring. The spring is trapped between the disk and a pair of cross-wires.

We have now appreciated that the spacers described in the prior art and those commercially available to date, whilst of some merit, are not entirely satisfactory. In particular, spacers comprising inhalation valves of the elastomeric slit membrane type have the disadvantage that the valve may not open sufficiently, particularly if the spacer is being used by an infant or young child. In such a valve there is variable opening of the valve depending on the inspiratory airflow.

We have now appreciated the need for an improved spacer and have devised one which substantially overcomes the problems associated with the known devices.

Accordingly to the present invention, there is provided a spacer device (100) for the oral administration of a volatile medium containing a medicament, which device comprises a chamber (110) having an inlet (102) to admit a measured dose of medicament and an outlet (104) to be received in the mouth, wherein the spacer comprises a butterfly valve (106).

The valve acts as a barrier to exhalation.

Preferably, the butterfly valve comprises one or more flaps pivotally mounted on a valve seat, although other designs are possible. Preferably, the valve comprises two flaps, more preferably two substantially semi circular flaps. In one preferred embodiment, two flaps pivot about an axis which passes through, or close to, the centre of the flow path. Suitably, the axis is substantially perpendicular to the flow path. Preferably, each flap comprises projections, suitably two projections, which enable it to be mounted on the valve seat. The flap may, for example, rotate about an axis between two projections of the flap (as will be further appreciated from the accompanying drawings). Preferably the projections of the flaps locate in corresponding recesses in the valve seat and the flaps rotate about the projections. Preferably the projections provide the only connection or mounting point between the flaps and the remainder of the valve. Preferably, each projection and its corresponding recess is substantially cylindrical. The valve is configured so as to permit air flow in one direction only—that is, out of the chamber. Preferably, the overall shape of the valve is circular.

The spacer device of the present invention is of a simple and efficient design, and this facilitates manufacture. The use of a butterfly valve allows operation of the spacer even at very low flow rates. Efficient operation is also achieved because the valve opens completely, even at low flow rates, rather than partially. We have also found that, in the context of spacers, butterfly valves do not show increased wear and tear over other types of valves, and also have the advantage of being easier to clean in situ, particularly when used with a spacer of our preferred design. The butterfly valve preferably emits an audible sound upon operation (usually upon closure) and this provides a useful indication to the user. The audible feature can be an integral part of the functioning of the valve—that is, no separate sonic signalling device is required—so this provides a further advantage.

The valve can be made of any suitable material, for example any rigid plastic or an antistatic non-metallic material, such as an antistatic plastic material, may be used. We prefer to use an acetyl copolymer (for example Delrin). Polyamide may also be used.

It is preferred to use a butterfly valve which operates at a very low expiratory flow rate. The valve preferably operates at an expiratory air flow of less than 25 ml per minute, for example from 15 ml to 25 ml per minute, or less. This enables closure of the valve even upon slight expiration by the user.

Preferably, the valve also operates at a very low inspiratory flow rate. The valve preferably operates at an inspiratory air flow rate of less than 25 ml per minute, for example from 15 ml to 25 ml per minute, or less. Complete valve opening thus occurs even upon slight inspiration by the user.

The butterfly valve is preferably positioned in close proximity to the outlet. Whilst the spacer device may be of various shapes and constructions, it is particularly preferred to use a spacer comprising two frustoconical members assembled together coaxially at their divergent ends, the inlet and outlet being respectively at opposed convergent ends. Further details of such a spacer can be obtained by reference to our publication WO 00/33902. Thus, when using such a spacer, the butterfly valve is preferably positioned at, or close to, the convergent end of the frustoconical member forming the outlet.

Preferably, the butterfly valve emits an audible sound upon closure.

The outlet of the spacer preferably comprises means for guarding the valve so as to prevent damage to the valve. Suitably, the guarding means may comprise a grid spanning the outlet although various designs are possible.

The chamber of the spacer is preferably made of an antistatic non-metallic material, for example an antistatic plastic material, for example polyamide. The valve may also be made of the same material.

For a spacer comprising two frustoconical members as described above, preferably the divergent end of one member is received in the divergent end of the other member so as to provide a substantially air-tight seal. Preferably, the divergent ends have complementary stepped surfaces to provide a close air-tight fit. It is also preferred to provide locking means to lock the two members together in assembled condition.

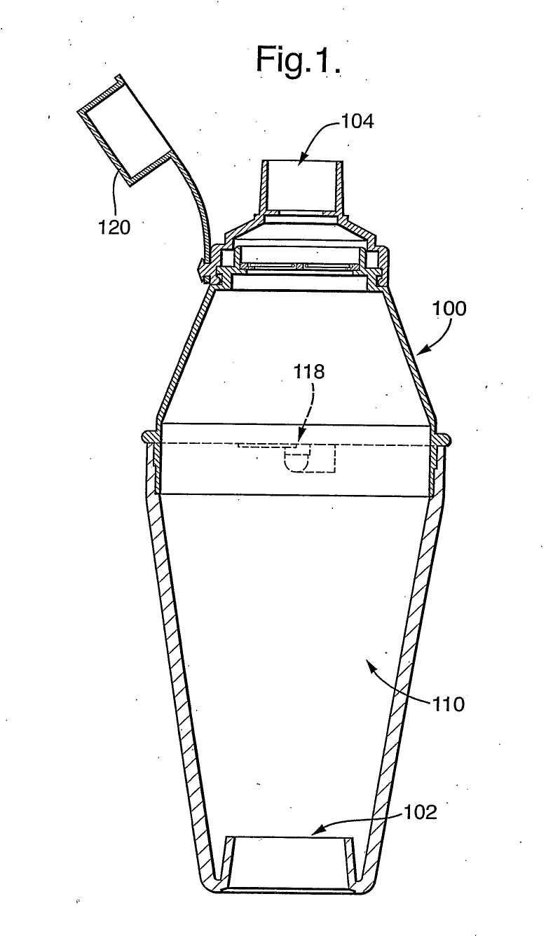

Preferably, the inlet of the spacer projects inside the chamber. For example, one embodiment of this is illustrated in FIGS. 1 and 4, which show inlet 102.

The spacer preferably also comprises one or more airvents. These are preferably located at the convergent end of the chamber member bearing the outlet, and allow for exit of any exhaled air to the atmosphere.

The spacer device preferably comprises an outlet having a mouthpiece. The mouthpiece preferably further comprises a cap, which may be attached or removable, as illustrated, for example, in FIG. 1. The cap preferably also comprises a connecting portion which attaches to a surface of the spacer, such that even when removed from the outlet the cap remains attached to the body of the spacer device.

The invention also provides the combination of an inhaler for dispensing a measured dose of medicament in a volatile medium and a spacer device according to the present invention.

The use of a spacer device according to the present invention for the inhalation of a medicament in a volatile medium is also provided.

To illustrate the invention, a preferred embodiment thereof will now be described with reference to the accompanying drawings in which:

FIG. 1 shows a general perspective view of a spacer device according to present invention.

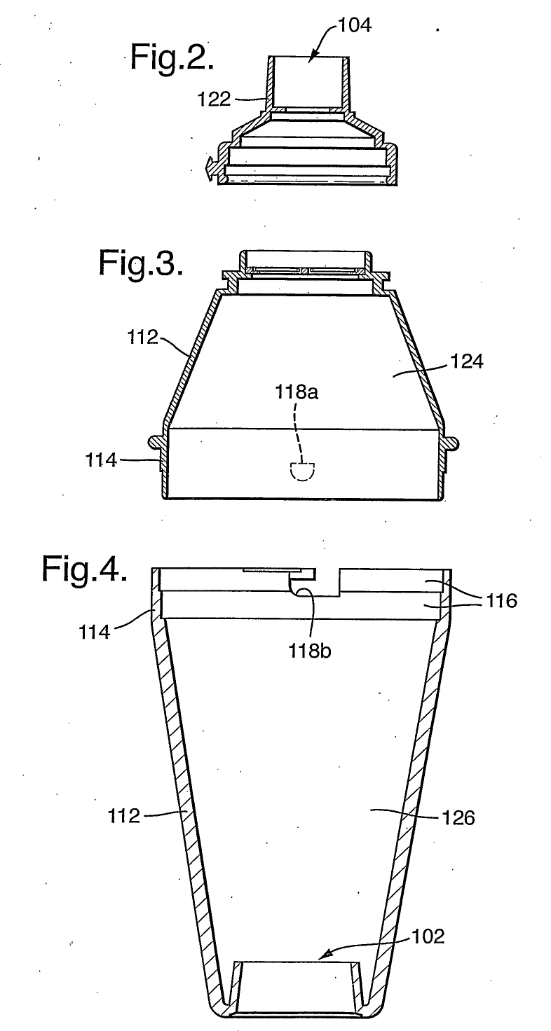

FIG. 2 shows a sectional view of the mouthpiece assembly of the spacer of FIG. 1.

FIG. 3 shows a sectional view of the top of the spacer of FIG. 1.

FIG. 4 shows a sectional view of the bottom of the spacer of FIG. 1.

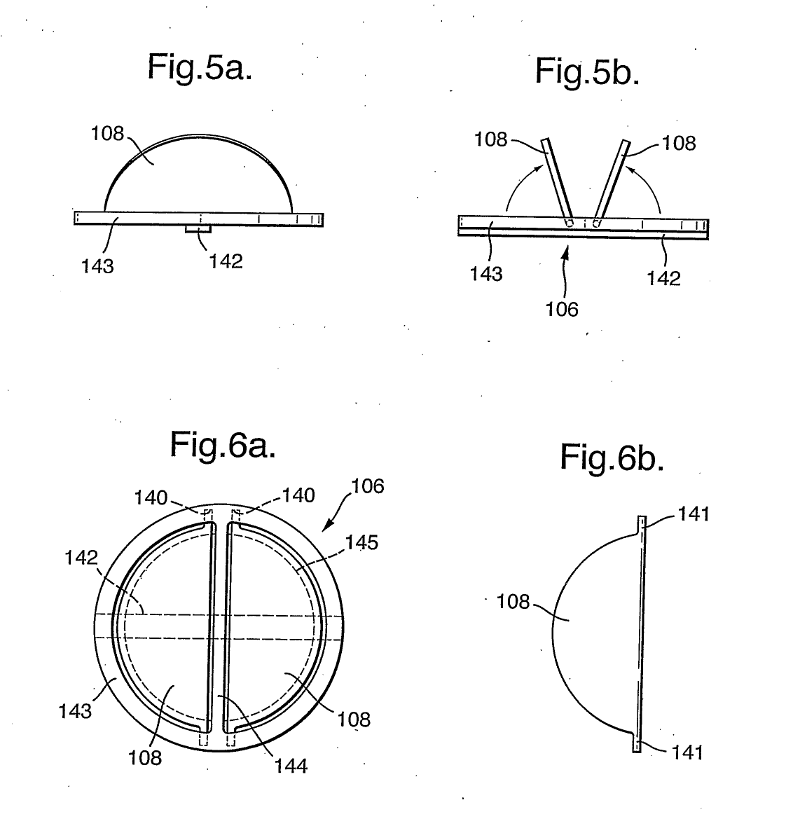

FIG. 5 shows a top view of the valve.

FIG. 6a shows a front view of a preferred butterfly valve; while FIG. 6b shows a front view of one or the semicircular flaps used in the butterfly valve of FIG. 6a.

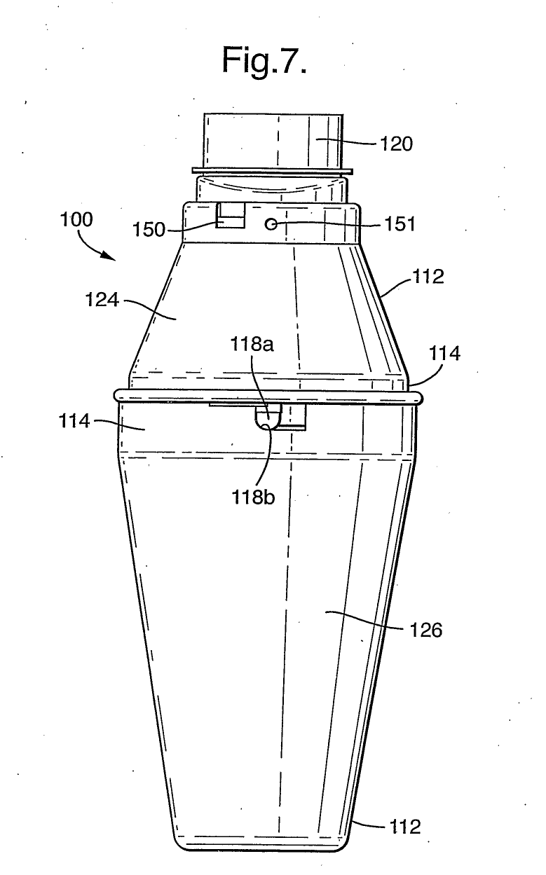

FIG. 7 shows a general perspective view of a spacer device according to the present invention.

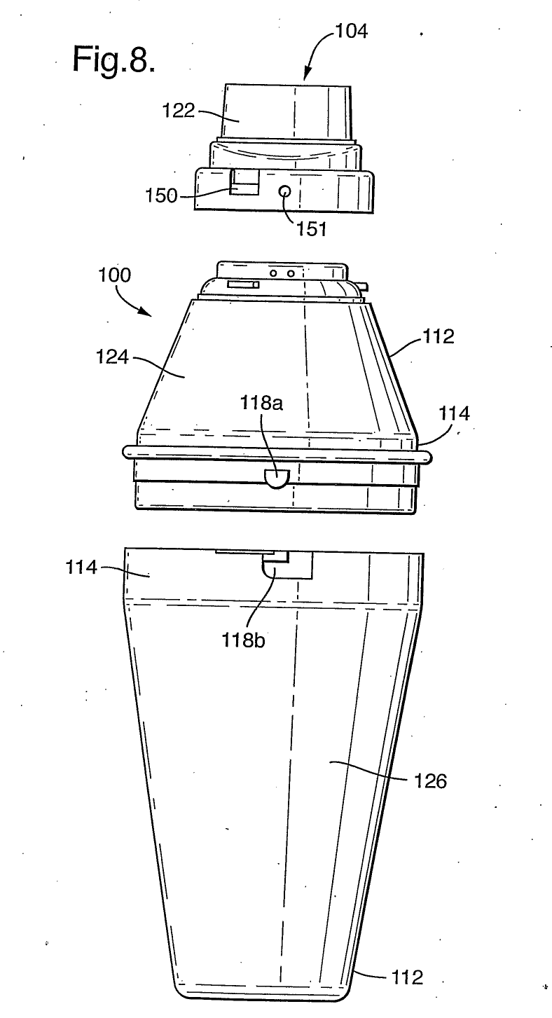

FIG. 8 shows an exploded general perspective view of the spacer device of FIG. 7.

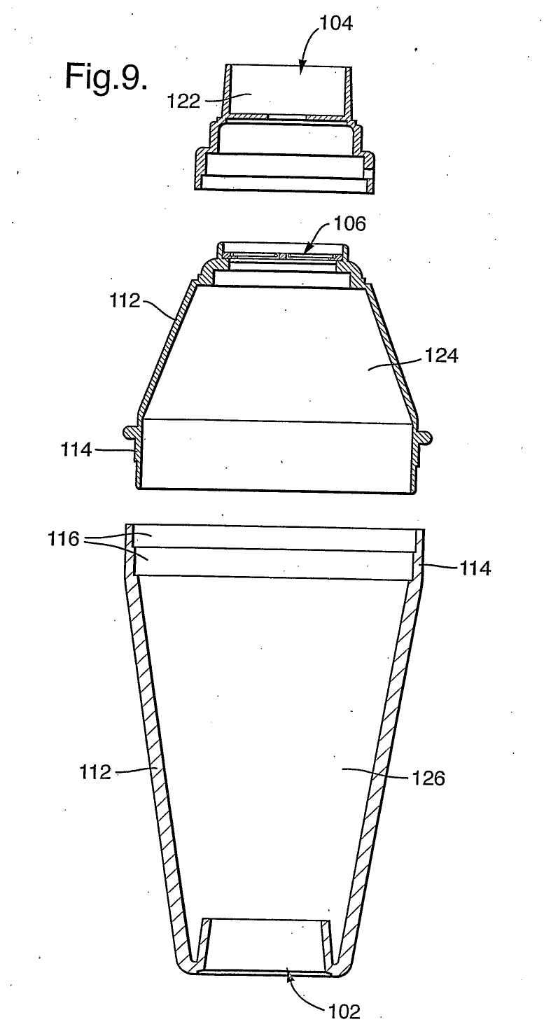

FIG. 9 shows a cross-sectional view of the spacer device of FIG. 8.

The butterfly valve (106) functions in such a manner that when the medicament is inhaled by the user, the flaps of the butterfly valve open as shown in FIG. 5 and allow the medicament to pass thorough the outlet into the patient's mouth. When there is exhalation by the user into the spacer device, even at substantially low expiration airflow, the flaps (108) of the butterfly valve (106) close the outlet and act as a barrier to exhalation. This prevents the chamber-containing medicament from being diluted with the moist air. While closing the outlet, the valve creates a ‘tap’ sound that provides an audio feedback to the patient. The butterfly valve functions at a very low expiratory air flow of 15 to 25 ml per minute.

The chamber (110) of the spacer device (100) has two conical members named spacer top (124) and spacer bottom (126) each having a convergent (112) and a divergent end (114); said members are assembled at divergent ends (114). The divergent ends (114) of the said conical members are provided with stepped rings (116) that enables the assembly of the said conical chambers and provides an airtight joint. The inlet (102) for receiving the medicament from the inhaler or the like and outlet (104) to deliver the medicament are placed on the opposite convergent ends (112) of said conical members so as to provide substantially good drug delivery.

A locking means (118) is provided on the said conical members for locking the assembled conical members. According to the present invention, a small projection (118a) is provided on one of the conical chambers and the other conical chamber is provided with a slit (118b) that fits the projection thereby providing a lock.

Further, the spacer device is provided with a cap (120) that covers the outlet of the spacer device. The cap is preferably attached to the spacer device.

According to an embodiment of the present invention, the outlet is provided with a mouthpiece (122). The mouthpiece is covered by the cap 120 as shown in FIGS. 1 and 7.

The mouthpiece assembly shown generally in FIG. 2 comprises the butterfly valve (106) together with associated guarding means (not shown in FIG. 2).

The spacer device of the invention can be made in accordance with known techniques, as will be clear to those skilled in the art.

In use, inlet 102 is connected to an aerosol medicament reservoir, for example, and a dose of inhalant medicament is pumped into chamber 110. The patient then places outlet 104 in the mouth and inhales steadily to draw the medicament into the lungs.

FIGS. 6a and 6b show a preferred embodiment of the valve (106). Valve (106), comprising a valve seat (143) with central spindle (144), supports two semi circular flaps (108) having projections (141) as shown in FIG. 6b. The projections (141) locate within recesses (140) in seat (143) such as to allow rotational movement of the flaps (108) (which may be better appreciated from FIG. 5). Preferably, recesses (140) and corresponding projections (141) are substantially cylindrical. Each flap (108) pivots very freely on the projections (141), thus enabling operation of the valve at low flow rates. When in the closed position, the flaps (108) seal against circumferential annulus (145) which may be integrally formed with the seat (143). Cross-member (142) may, for example, optionally be provided to give structural support.

FIG. 7 additionally shows airvent (150) and notch (151) which may serve as a point of attachment of the cap (120) to the device (as shown in FIG. 1).

FIGS. 8 and 9 provide further illustration of the various features described above.

The invention is further illustrated by the following Example.

### EXAMPLE 1

### Fine Particle Dose (FPD) by Cascade Impactor Using Different Spacers

### Using Flixotide Evohaler-250 mcg/Spray

| | | Aerochamber Plus | |
|---|---|---|---|
| | Spacer with valve | spacer | |
| | According to the | (Trudell Medical | Volumatic spacer |
| | present invention | International) | (Glaxo) |
| FPD (mcg) | 112.28 | 55.18 | 12.59 |

### T1/2 Life of Dose in Spacer of Present Invention and Spacer Similar to that of U.S. Pat. No. 5,042,467

### Using Salbutamol Inhaler

| | Spacer with valve | |
|---|---|---|
| | according to the | Spacer similar to |
| | present invention | U.S. Pat. No. 5,042,467 |
| FPD (mcg) after 2 sec | 65.01 | 10.5 |
| lag time | | |
| FPD (mcg) after 45 sec | 40.25 | 2.7 |
| lag time | | |

Lag time is the time between actuation of the dose into the spacer and dosing into the apparatus for testing (Anderson Cascade Impactor)

T1/2 of the dose in the spacer is the quantitative measure of the time up to which 50% of the respirable dose is available for inhalation. It is the time taken for reduction of the FPD value to 50% of the original. The results show the FPD value is maintained at a much higher level for longer with a spacer device according to the invention.

1. A spacer device (100) suitable for use with a metered dose inhaler for the oral administration of a volatile medium containing a medicament, which device comprises a chamber (110) having an inlet (102) to admit a measured dose of medicament and an outlet (104) to be received in the mouth, wherein the spacer comprises a breath-operated butterfly valve which comprises one or more flaps pivotally mounted on a valve seat (106).

2. The device according to claim 1 wherein the butterfly valve is in proximity to the outlet.

3. The device according to claim 1 wherein the valve operates at a low expiratory air flow of 15 to 25 ml per minute or less.

4. The device according to claim 1 wherein the valve operates at a low inspiratory air flow of 15 to 25 ml per minute or less.

5. The device according to claim 1 wherein the valve emits an audible sound upon closure.

6. The device according to claim 1 wherein the outlet comprises means for guarding the valve.

7. The device according to claim 6 wherein the means for guarding the valve comprises a grid spanning the outlet.

8. The device according to claim 1 wherein the chamber is made of an antistatic non-metallic material.

9. The device according to claim 1 wherein the chamber is made of an antistatic plastic material.

10. The device according to claim 9 wherein the chamber is made of polyamide.

11. The device according to claim 1 wherein the valve is made of any rigid plastic or antistatic non-metallic material.

12. The device according to claim 1 wherein the valve is made of an acetyl copolymer or polyamide

13. The device according to claim 1 wherein the chamber comprises two frustoconical members assembled together coaxially at their divergent ends, said inlet and outlet being respectively at the opposed convergent ends.

14. The device according to claim 13 wherein the divergent end of one member is received in the divergent end of the other member to provide a substantially air-tight seal.

15. The device according to claim 14 wherein the said divergent ends have complementary stepped surfaces to provide a close air-tight fit.

16. The device according to claim 13 wherein locking means are provided to lock the two members together in assembled condition.

17. The device according to claim 1 wherein the inlet projects inside the chamber.

18. The device according to claim 1 wherein the outlet comprises a mouthpiece.

19. The device according to claim 18 wherein the mouthpiece further comprises a cap.

20. The device according to claim 19 wherein the cap is attached to the device or is a removable cap.

21. A combination of an inhaler for dispensing a measured dose of a medicament in a volatile medium and a spacer device according to claim 1.

22. A method comprising using a device according to claim 1 for the inhalation of a medicament in a volatile medium.
